(19) United States
(12) Patent Application Publication
(10) Pub. No.: US 2004/0220479 A1
(43) Pub. Date: Nov. 4, 2004

(54) Folded optics in a laser imaging apparatus with an ergonometric tabletop

(76) Inventors: Robert H. Wake, Cooper City, FL (US); Vicente Magraner, Miami, FL (US)

Correspondence Address: SHLESINGER, ARKWRIGHT & GARVEY LLP, 3000 South Eads Street, Arlington, VA, 22202, US

(21) Appl. No.: 10/832,282

(22) Filed: Apr. 27, 2004

Related U.S. Application Data
(60) Provisional application No. 60/466,062, filed on Apr. 29, 2003.

(51) Int. Cl. A61B 6/00

(52) U.S. Cl. 600/476

(57) ABSTRACT

A scanner for a laser imaging apparatus comprises a tabletop having an opening in which a breast to be scanned is disposed; a reflector ring disposed around the opening below the tabletop, the ring having a reflective surface facing the opening and disposed at an angle downwardly away from the opening; a laser beam directed upwardly below the tabletop toward the reflective surface such that the beam is reflected across the opening toward an opposite portion of the reflective surface, the beam going across the opening defining a slice plane; a plurality of collimators including vertical channels directed toward the reflective surface such that light exiting from the breast within the field of view of the collimators is reflected by the reflective surface down through the vertical channels; a plurality of optical detectors, each detector being disposed below the respective vertical channels. The laser beam, collimators and detectors are adapted to be orbited at the same time around the breast about an orbital axis through the opening. Each of said detectors is configured to simultaneously detect light exiting the breast being scanned within the respective field-of-view of each collimator.

### RELATED APPLICATIONS

This application claims the benefit of U.S. Provisional Application Serial No. 60/466,062, filed Apr. 29, 2003, hereby incorporated by reference.

### FIELD OF THE INVENTION

The present invention relates generally to a diagnostic medical imaging apparatus that employs a near-infrared laser as a radiation source and a detector array with restricted fields of view directed to their own patches of surface of the object being scanned to simultaneously detect the intensity of light exiting from the object for the purpose of reconstructing cross-sectional images of the object, and more particularly to folded optics in such a laser imaging apparatus with an ergonometric tabletop.

### BACKGROUND OF THE INVENTION

Cancer of the breast is a major cause of death among the American female population. Effective treatment of this disease is most readily accomplished following early detection of malignant tumors. Major efforts are presently underway to provide mass screening of the population for symptoms of breast tumors. Such screening efforts will require sophisticated, automated equipment to reliably accomplish the detection process.

The x-ray absorption density resolution of present photographic x-ray methods is insufficient to provide reliable early detection of malignant tumors. Research has indicated that the probability of metastasis increases sharply for breast tumors over 1 cm in size. Tumors of this size rarely produce sufficient contrast in a mammogram to be detectable. To produce detectable contrast in photographic mammograms 2-3 cm dimensions are required. Calcium deposits used for inferential detection of tumors in conventional mammography also appear to be associated with tumors of large size. For these reasons, photographic mammography has been relatively ineffective in the detection of this condition.

Most mammographic apparatus in use today in clinics and hospitals require breast compression techniques which are uncomfortable at best and in many cases painful to the patient. In addition, x-rays constitute ionizing radiation which injects a further risk factor into the use of mammographic techniques as most universally employed.

Ultrasound has also been suggested as in U.S. Pat. No. 4,075,883, which requires that the breast be immersed in a fluid-filled scanning chamber. U.S. Pat. No. 3,973,126 also requires that the breast be immersed in a fluid-filled chamber for an x-ray scanning technique.

In recent times, the use of light and more specifically laser light to noninvasively peer inside the body to reveal the interior structure has been investigated. This technique is called optical imaging. Optical imaging and spectroscopy are key components of optical tomography. Rapid progress over the past decade has brought optical tomography to the brink of clinical usefulness. Optical wavelength photons do not penetrate in vivo tissue is a straight line as do x-ray photons. This phenomenon causes the light photons to scatter inside the tissue before the photons emerge out of the scanned sample.

Because x-ray photon propagation is essentially straight-line, relatively straight forward techniques based on the Radon transform have been devised to produce computed tomography images through use of computer algorithms. Multiple measurements are made through 360° around the scanned object. These measurements known as projections are used to backproject the data to create an image representative of the interior of the scanned object.

In optical tomography, the process of acquiring the data that will ultimately be used for image reconstruction is the first important step. Light photon propagation is not straight-line and techniques to produce cross-section images are mathematically intensive. To achieve adequate spatial resolution, multiple sensors are employed to measure photon flux density at small patches on the surface of the scanned object. The volume of an average female breast results in the requirement that data must be acquired from a large number of patches. The photon beam attenuation induced by breast tissue reduces the available photon flux to a extremely low level and requires sophisticated techniques to capture the low level signals.

U.S. Pat. No. 5,692,511 discloses such a laser imaging apparatus. In this apparatus, the detector housings (collimators) are perpendicular to the orbit axis, therefore parallel to the patient's chest wall. The detector housings (collimators) for any given slice lie in a plane, the optical plane or slice plane. The detector array is consequently “planar”.

The use of a planar detector array dictates that the patient support surface (the tabletop) surrounding the breast be planar, flat. However, a more desirable patient support surface would allow vertical relief for the patient's shoulder, arms, other breast and head to provide comfort to the patient during scanning.

### OBJECTS AND SUMMARY OF THE INVENTION

It is an object of the present invention to provide a scanner structure that accommodates the raised and lowered support surfaces that support a patient's other body portions while her breast or other body portion is being scanned.

It is another object of the present invention to provide a scanner that uses folded optics to allow placement of the laser and the detectors some distance below the raised surfaces of the tabletop and yet allow scanning of the breast adjacent the raised surfaces.

It is a further object of the present invention to provide contiguous arrays of sensors around a scanned object wherein the sensors have a field of view restricted to the volume within a single slice plane and the sensors and radiation beam are translated to parallel slice planes, contiguous or separated, through a scanned object, with the scan planes being offset from the radiation beam plane.

It is another object of the present invention to provide one or more sensors positioned in a circle or arc around a scanned object that allow simultaneous acquisition of photon intensity data on the surface of a scanned object illuminated by one or more radiation beams while the sensors and radiation beam are rotated around the scanned object and the scan slice planes are offset from the radiation beam plane.

It is another object of the present invention to provide one or more sensors positioned in a circle or arc around a scanned object that allow simultaneous acquisition of photon intensity data on the surface of a scanned object illuminated by one or more radiation beams while the sensors and radiation beam are at one fixed location and the scan slice planes are offset from the radiation beam.

It is still another object of the present invention to provide a plurality of detectors to acquire photon transport data from tissue within multiple parallel planes separated by thicknesses to form multiple slices of a total volume.

It is another object of the present invention to provide a plurality of sensors to acquire photon transport data from tissue within multiple parallel planes separated by thicknesses to form slices of a total volume to reconstruct a 3-dimensional image of the interior of the scanned volume.

It is another object of the present invention to provide contiguous arrays of sensors around a scanned object and wherein the sensors have a field of view restricted to the volume within a single slice plane while the sensors and radiation beam are at one fixed point with respect to the scanned object.

It is another object of the present invention to provide contiguous arrays of sensors around a scanned object and wherein the sensors have a field of view restricted to the volume within a single slice plane while the sensors and radiation beam are rotated around the scanned object.

In summary, the present invention provides a scanner for a laser imaging apparatus, comprising a tabletop having an opening in which a breast to be scanned is disposed; a reflector ring disposed around the opening below the tabletop, the ring having a reflective surface facing the opening and disposed at an angle inclined toward the opening; a laser beam originating below the tabletop and directed upwardly toward the reflective surface such that the beam is reflected across the opening toward an opposite portion of the reflective surface, the beam going across the opening defining a slice plane; a plurality of collimators including vertical channels directed toward the reflective surface such that light exiting from the breast within the field of view of the collimators is reflected by the reflective surface down through the vertical channels; a plurality of optical detectors, each detector being disposed below the respective vertical channels. The laser beam, collimators and detectors are adapted to be orbited at the same time around the breast about an orbital axis through the opening. Each of said detectors is configured to simultaneously detect light exiting the breast being scanned within the respective field-of-view of each collimator.

These and other objectives of the present invention will become apparent from the following detailed description.

### BRIEF DESCRIPTIONS OF THE DRAWINGS

FIG. 1 is a schematic side elevational view of a scanning apparatus with a planar detector array configuration, showing a prone patient positioned for an optical tomographic study, with one breast pendent within the scanning chamber.

FIG. 2 is a schematic top view of the scanning chamber of FIG. 1, showing the planar detector array, consisting of a plurality of detectors disposed around an object being scanned and a laser light source.

FIG. 3 is a schematic cross-sectional view through the planar detector array of FIG. 2, showing the laser light source and the detectors.

FIG. 4 is a schematic side elevational view of a scanning apparatus with a tabletop having vertical relief around the breast and showing a prone patient positioned for an optical tomographic study, with one breast pendent within the scanning chamber.

FIG. 5A is a perspective top view of the scanning apparatus of FIG. 4, showing the tabletop with vertical relief around the breast and the scanning chamber.

FIG. 5B is a top plan view of FIG. 5A.

FIG. 6 is a block diagram of the data acquisition system that supports the detector array of FIGS. 2 and 3.

FIG. 7 is a cross-sectional view through the folded-optics detector array of FIG. 4, showing the laser light source, a conical folding mirror and detectors for two slices.

FIG. 8 is a cross-sectional view through the folded-optics detector array of FIG. 4, showing the laser light source, a conical folding mirror and detectors for five slices.

FIG. 9 is a cross-sectional view through the folded-optics detector array of FIG. 4, showing the laser light source, a conical folding prism and detectors for two slices.

FIG. 10 is a cross-sectional view through the folded-optics detector array of FIG. 4, showing the laser light source and detectors for two slices, employing optic fibers to achieve the folding function.

### DETAILED DESCRIPTION OF THE INVENTION

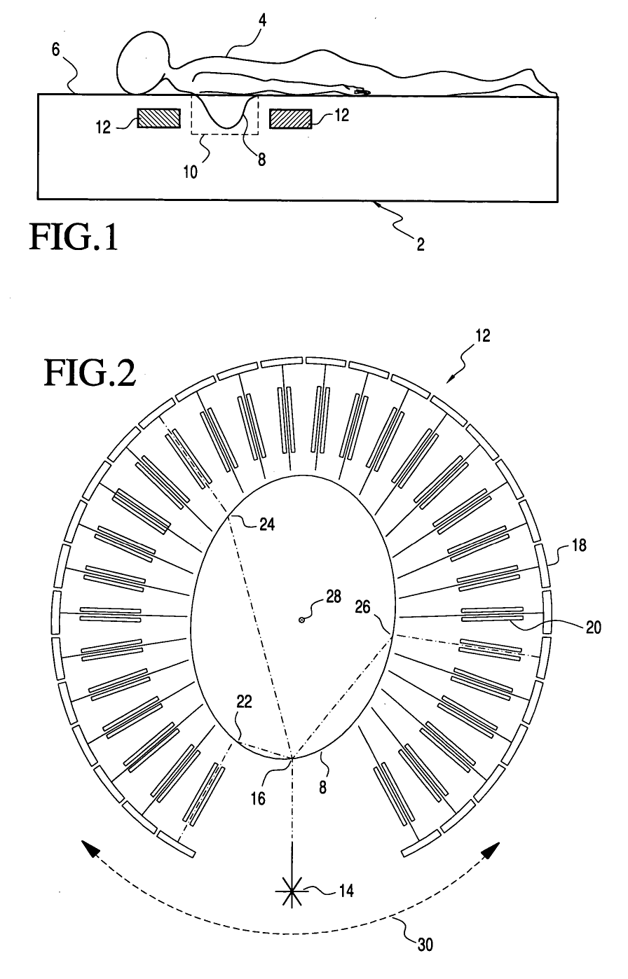

Referring first to FIG. 1, a scanning apparatus 2, as described in U.S. Pat. Nos. 5,692,511 and 6,100,520, supports a prone patient 4 on an essentially flat top surface 6. The patient's breast 8 is pendent within a scanning chamber 10, around which orbits a planar detector array 12. The planar detector array 12 orbits typically 360° around the vertical axis of the scanning chamber 10 and increments vertically between orbits to image successive slice planes. This is repeated until all the slice planes of the breast have been scanned. Since the surface 6 is a single level, flat surface, the patient's head and shoulder tend to contact the table surface, causing discomfort and lifting the breast somewhat out of the scanning chamber.

FIG. 2 shows a top view of the planar detector array 12 from FIG. 1. A laser source 14 generates a laser beams that impinges on the scanned object (breast) 8 at a point 16. A plurality of detectors 18 defines an arc surrounding the scanned object. A collimator 20 defines each detector's field of view to a small area on the surface of the scanned object. Light enters the scanned object at point 16 and exits at every point on its circumference. Three exit points 22, 24 and 26 are shown, corresponding to three detectors. The entire mechanism rotates around the center-of orbit rotation 28, as indicated by the curved double-headed arrow 30.

In the preferred implementation, every detector in the array is collimated, aiming at the center of orbit rotation 28. The laser source also points toward the center of rotation. The detectors are spaced at equal angular increments around the center of rotation. The orbit rotation is preferably alternately 360° clockwise for one (horizontal) slice plane, and 360° counterclockwise for the next slice plane.

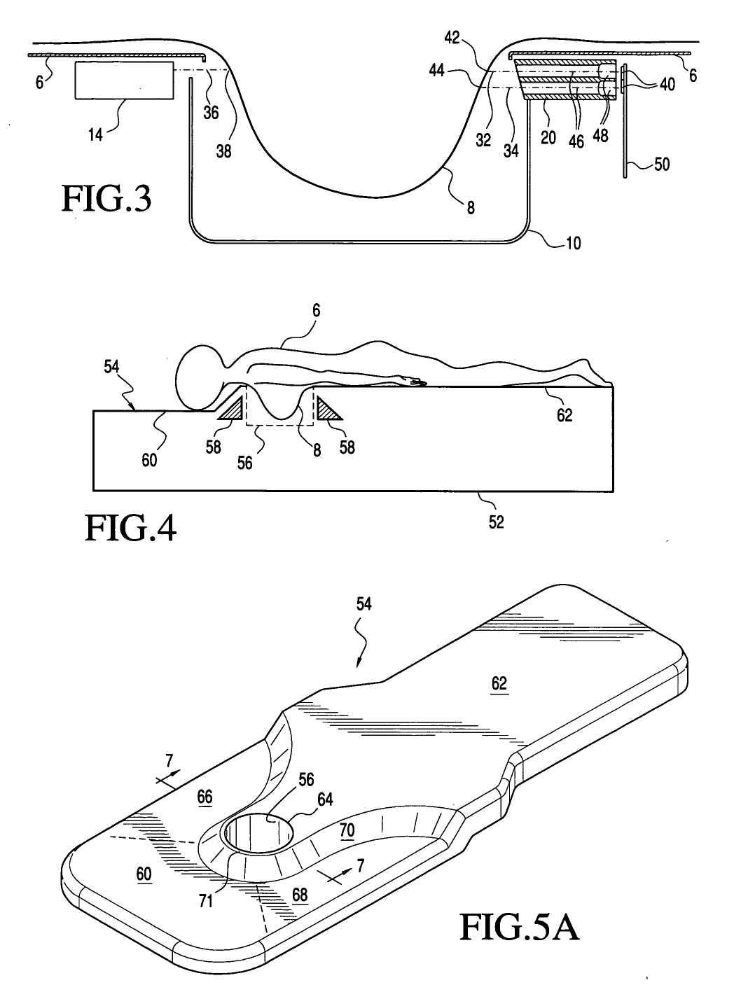

FIG. 3 shows a vertical cross-section through the planar detector array 12 of FIG. 2. The planar detector array 12 is shown as simultaneously imaging two adjacent slices 32 and 34, though any number of slices can be imaged simultaneously, as disclosed in U.S. Pat. No. 6,100,520. The patient's breast 8 is pendent within the scanning chamber 10. The patient is supported by the scanning apparatus' tabletop surface 6. The laser 14 projects a coherent light beam 36 which impinges on the patient's breast at point 38.

Two photodetectors 40, one each from the two slice planes 32 and 34, are shown imaging points 42 and 44 on the breast for the upper and lower slices, respectively. The opaque collimator 20 is shown as a single physical entity with two collimating channels 46. The collimating channels 46 can be round, square, hexagonal, triangular or any other cross-sectional shape. The collimator 20 advantageously restricts the field of view of each detector assembly to a small, defined area on the surface of the scanned object. At the rear of each collimating channel is a lens 48, which focuses the light propagating down the collimating channel onto the photodetector 40. The lenses are shown as plano-convex, but can be biconvex or can be eliminated if the photodetector's active area were larger than the collimating channel's-cross-sectional area. The photodetectors are connected to a signal processing system 50 for amplification and analog-to-digital conversion.

The laser 14 can be a semiconductor diode laser, a solid-state laser or any other near-infrared light source. The photodetectors 40 can be photodiodes, avalanche photodiodes, phototransistors, photomultiplier tubes, microchannel plates or any other photosensitive device that converts incoming light photons to an electrical signal. The photodetectors provide the means for detecting the laser beam after passing through the breast.

FIG. 4 shows a schematic side elevational view of a scanning apparatus 52 with a tabletop surface 54 shaped so as to allow vertical relief for the patient's shoulder, arms, head and opposite breast. A prone patient 4 is positioned for an optical tomographic study, with one breast 8 pendent within a scanning chamber 56. A folded-optics detector array 58, shown schematically, orbits typically 360° around the vertical orbital axis of the scanning chamber 56 and increments vertically downward between orbits to image successive slice planes. This is repeated until all the slice planes of the object have been scanned.

The tabletop surface 54 has a lower level surface 60 and a higher level surface 62. The lower level surface 60 advantageously provides relief and support for the patient's shoulder, arms, head and opposite breast. The higher level surface 62 advantageously provides support for the patient's lower body and legs.

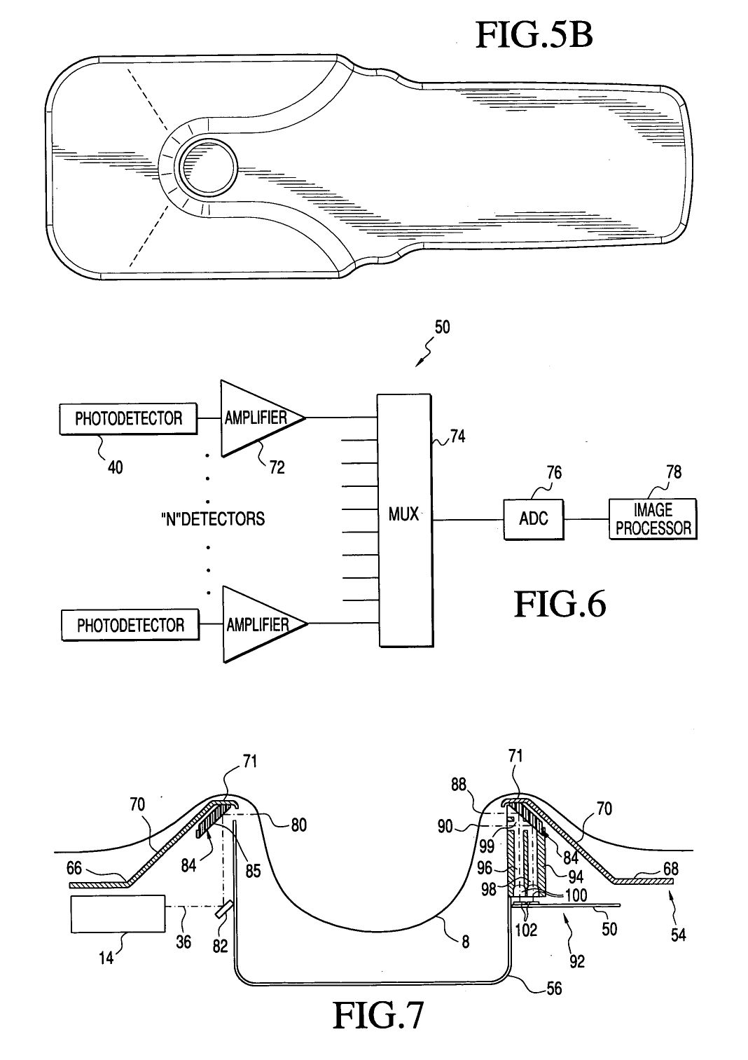

FIG. 5A shows the scanning apparatus tabletop 54 in perspective. The patient's breast 8 would be pendent in the scanning chamber 56. The patient's torso and legs are supported by the surface 62, which is advantageously at the same level as the opening 64 of the scanning chamber 56. The surface 60 supports the patient's head, advantageously allowing the head to be positioned below the top of the scanning chamber for comfort. Assuming the patient's left breast is positioned in the scanning chamber 56, surface 66 advantageously provides relief for the patient's right breast and surface 68 provides relief for the patient's left shoulder and a resting place for the patient's left arm. The roles of the surfaces 66 and 68 are reversed for scanning the right breast. The tabletop 54 is preferably symmetrical in plan-view, as shown in FIG. 5B.

Surfaces 60, 66 and 68 are at the same level in the preferred embodiment, approximately 7 centimeters below the rim of the scanning chamber 56. However, it should be understood that these surfaces can be at different levels. A transition surface 70 between the higher level surface 62 and the lower level surfaces 60, 66 and 68 is preferably slanted or ramped to provide room underneath for the detector array 58. The surface 62 preferably tapers toward the opening 64. The transition surface 70 also provides support for parts of the patient's body immediately adjacent the breast being scanned. A horizontal flange or lip 71 around the opening 64 provides further comfortable support to the peripheral base area of the breast being scanned.

The preferred embodiment has the patient lying prone with the breast pendent in the scanning chamber. Although the tabletop is shown horizontal for a patient in prone position, it should be understood that the tabletop can be in any position.

FIG. 6 shows the signal processing system 50. A plurality of photodetectors 40 are connected to a plurality of amplifiers 72. In the preferred embodiment, the photodetectors are photodiodes and the amplifiers are integrators. The amplifiers are connected to a multiplexer (MUX) 74 which presents one of “N” amplifier outputs to an analog-to-digital converter (ADC) 76. The digital output of the ADC is connected to an image processor 78, typically a general-purpose computer. The image processor performs the reconstruction computations to create cross-sectional images from the projection data collected by the scanning apparatus. Multiple MUXes and ADCs can be employed in order to decrease the data acquisition time.

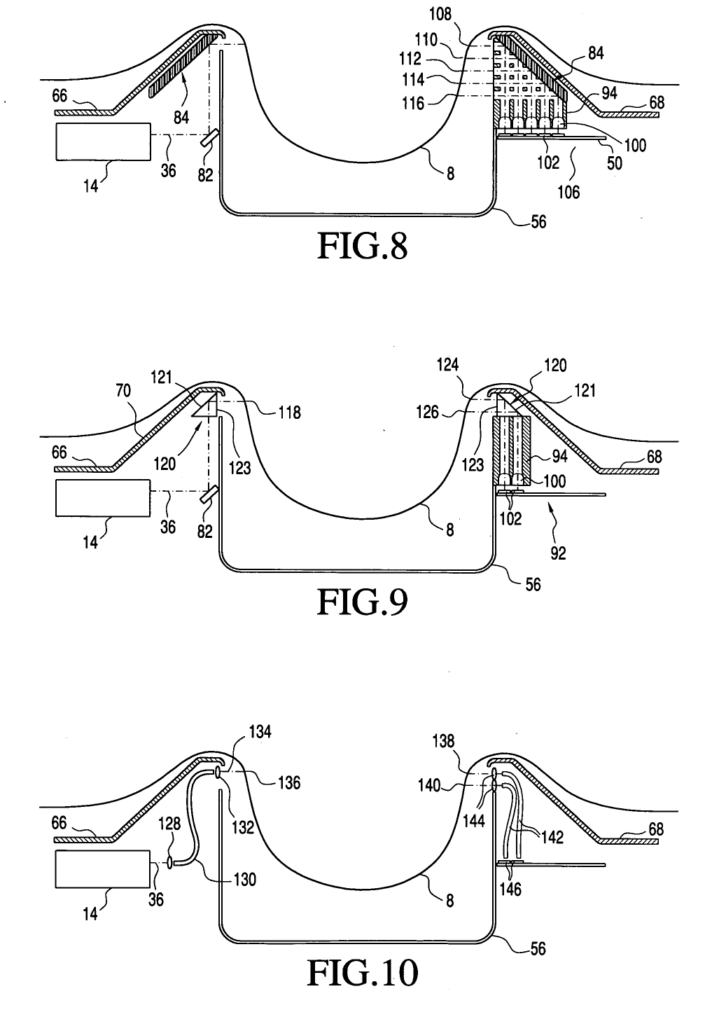

FIG. 7 shows a detailed vertical cross-sectional view along line 7-7 of FIG. 5 of the folded-optics detector array 58 shown schematically in FIG. 4. The folded-optics detector array is shown as simultaneously imaging two adjacent slices, the preferred embodiment, though any number of slices can be imaged simultaneously, such as five slices shown in FIG. 8. The patient's breast 8 is pendent within the scanning chamber 56. The patient is supported by the scanning apparatus' tabletop 54. The lower surfaces 66 and 68 are shown supporting the patient's left shoulder and the right breast (the left breast is shown within the scanning chamber). The laser 14 projects a coherent light beam 36 which impinges on the patient's breast at point 80 after reflecting from a planar turning mirror 82 and a conical mirror 84. The planar turning mirror 82 is commonly used. The conical mirror 84 is a ring around the scanning chamber 56 and is a segment, a frustum of a hollow cone with the inside 45° conical surface 85 being reflective. The same conical mirror 84 reflects light emitted from the breast at points 88 and 90 into “N” number of two-detector assemblies 92, which detect light coming from the upper and lower slices, respectively. The detector assemblies 92 are arranged in a circle or arc, with their longitudinal axes through the vertical channels all pointing upwards toward the conical mirror 84. The slanting surface 70 advantageously provides room for the conical mirror 84 and the collimators 94.

The conical mirror 84, in the preferred embodiment, is a diamond-turned aluminum mirror with a high-reflectivity gold plating on the inside conical surface 85. Alternatively, it can be polished glass or plastic with a reflective coating, or any other reflective material capable of being formed into a conical shape. The laser turning mirror 82 can be eliminated with the laser 14 projecting vertically onto the conical mirror 84.

The detector assemblies 92 consist of an opaque collimator 94, shown as a single physical entity with two collimating channels 96 and 98. The collimating channels are folded 90° at 99 by the conical mirror 84 and actually intersect each other, forming horizontal and vertical channels. The intersecting collimating channels, which allow the light to intersect, are not of concern, since light at these power levels, in air, does not interact with itself. There is no interference between the light from areas 88 and 90 although their paths cross. The collimating channels can be round, square, hexagonal, triangular or an other cross-sectional shape. The collimator restricts the fields of view of each detector assembly to a small, defined area on the surface of the breast 8, the scanned object.

At the rear or bottom of each vertical collimating channel is a lens 100, which focuses the light propagating down the collimating channel onto the photodetector 102 located below the exit ends of the vertical channels. The lenses are shown as plano-convex, but can be biconvex or can be eliminated if the photodetector's active area were larger than the collimating channel's cross-sectional area. The photodetectors 102 are connected to the signal processing system 50, providing amplification and analog-to-digital conversion, as shown in FIG. 6.

The laser 14 and photodetectors 102 are the same as described for the planar detector configuration.

FIG. 8 shows a five-detector assembly 106, using the conical mirror 84 of FIG. 7. The laser 14 projects a coherent light beam 36 which impinges on the patient's breast 8 after reflecting from the planar turning mirror 82 and the conical mirror 84. The same conical mirror 84 reflects light emitted from the breast at points 108, 110, 112, 114 and 116 into “N” number of the five-detector assemblies 106, advantageously allowing the simultaneous imaging of five consecutive slices. The detector assemblies 106 are arranged in a circle or arc around the scanning chamber 56. It should be understood that the horizontal channels form a series of arcs around the opening of the scanning chamber, each arc being disposed vertically below the topmost arc. It should also be understood that the vertical channels similarly form a series of arcs around the opening, where each arc is larger than an adjacent arc nearer to the opening.

FIG. 9 shows an alternative to the conical mirror 84 for folding the optical path of both the laser beam 36 and the detectors 102. The laser 14 projects a coherent light beam 36 which impinges on the patient's breast 8 at 118 after reflecting from the planar turning mirror 82 and a conical prism 120. The conical prism 120 is a ring around the scanning chamber 56 and is a right triangle swept into a circle. Its outside surface 121 is preferably conical; its inside surface 123 is preferably cylindrical. Its cross-section is the classic 45° prism, which reflects light 90° by total internal reflection. The same conical prism 120 reflects light emitted from the breast at points 124 and 126 into “N” number of two-detector assemblies 92 imaging the upper and lower slices, respectively. The detector assemblies are arranged in a circle or arc, with the longitudinal axes of the vertical collimator channels all pointing upwards toward the conical prism 120. The prism 120 may be made of optical glass, sapphire, quartz, various plastics or any other material with a high transmission of near-infrared light.

FIG. 10 shows an alternative to the conical mirror 84 or the prism 120 for folding the optical path of both the laser and the detectors. The laser 14 projects a coherent light beam 36 which is coupled by lens 128 into an optical source fiber 130. Lens 132 collimates the light from the source fiber 130 and projects a parallel beam 134 which impinges on the breast at point 136. Light emitted from the breast at points 138 and 140 is focused into optical detector fibers 142 by lenses 144. The detector fibers 142 receive the light at their entry ends and conduct the light to detectors 146 located near their exit ends. Additional lenses may be interposed between the detector fibers 142 and the detectors 146, depending on the relative size of the optical fiber and detector. Lens 128 may be eliminated depending on the relative size of the optical fiber 130 and the beam diameter from the laser 14. Lenses 132 and/or 144 may be eliminated, depending on the size and numerical aperture of the optical fibers and their proximity to the breast. It should be understood that the entry ends of the optic fibers 142 form a series of arcs around the opening, where each arc is disposed below the topmost arc.

A number of planar mirrors can be employed alternatively to the conical mirror 84, with the mirrors all at 45° and butted end-to-end forming an “N”-sided polygon. The more mirrors are used, the closer the approximation would be to the single-piece conical mirror. Similarly, a number of 45° prisms, butted end-to-end to form a polygonal ring, can be used in place of the single conical prism.

The mirror or the prism alters the light paths from the breast from the horizontal scan plane to the vertical longitudinal axes of the detector assemblies. The “folding angle” is disclosed as 90°, but it should be understood that other folding angles can be employed. Further, the scan plane need not be horizontal and the orbital axis vertical. In an optical scanner used for head imaging, for example, the scan plane can be vertical, or nearly so, with an essentially horizontal orbital axis. It should also be understood that the mirrors, prism or the optic fibers provide the means for folding and directing the laser beam across the opening from a location below the scan plane.

The various collimators disclosed provide the means for restricting the field-of-view of each photodetector to a small area on the surface of the breast.

While this invention has been described as having a preferred design, it is understood that it is capable of further modification, uses and/or adaptations of the invention following in general the principle of the invention and including such departures from the present disclosure as come within known or customary practice in the art to which the invention pertains, and as may be applied to the essential features set forth, and fall within the scope of the invention or the limits of the appended claims.

We claim:

1. A scanner for a laser imaging apparatus, comprising:
a) a tabletop having an opening in which a breast to be scanned is disposed;
b) a reflector ring disposed around said opening below said tabletop, said ring having a reflective surface facing said opening and disposed at an angle inclined toward said opening;
c) a laser beam originating below said tabletop and directed upwardly toward said reflective surface such that said beam is reflected across said opening toward an opposite portion of said reflective surface, said beam going across said opening defining a slice plane;
d) a plurality of collimators including vertical channels directed toward said reflective surface such that light exiting from the breast within the field of view of said collimators is reflected by said reflective surface down through said vertical channels;
e) a plurality of optical detectors disposed below said scan plane and below the output end of the respective vertical channels;
f) said laser beam, said collimators and said detectors are adapted to be orbited at the same time around the breast about an orbital axis through said opening; and
g) each of said detectors are configured to simultaneously detect light exiting the breast being scanned within the respective field-of-view of each collimator.

2. A scanner as in claim 1, wherein said reflective surface is a conical surface angled at 45° from a vertical axis.

3. A scanner as in claim 1, wherein said reflective surface includes a plurality of planar mirrors butted end-to-end to form a polygon.

4. A scanner as in claim 1, wherein:
a) said ring is a prism in cross-section having an inclined; and
b) said inclined surface includes said reflective surface.

5. A scanner as in claim 4, wherein said inclined surface is at an angle of 45° from a vertical axis.

6. A scanner as in claim 4, wherein said inclined surface is a conical surface.

7. A scanner as in claim 4, wherein said ring includes a plurality of prisms joined end-to-end to form a polygon.

8. A scanner as in claim 4, wherein:
a) said collimators include horizontal channels having their front ends directed toward said opening and their rear ends directed toward said reflective surface; and
b) said vertical channels are aligned with respective horizontal channels via said reflective surface.

9. A scanner as in claim 1, wherein said vertical channels include respective lenses at their bottom ends to focus said laser beam to said respective detectors.

10. A scanner as in claim 1, wherein said vertical channels form a series of arcs around said opening, each arc being larger than an adjacent arc nearer to said opening.

11. A scanner as in claim 8, wherein said horizontal channels form a series of arcs around said opening, each arc being disposed vertically below a topmost arc.

12. A scanner as in claim 1, and further comprising:
a) a turning mirror disposed below said reflective surface; and
b) said laser beam is aimed at said mirror such that said laser beam is reflected upwardly toward said reflective surface and reflected across said opening.

13. A scanner for a laser imaging apparatus, comprising:
a) a tabletop having an opening in which a breast to be scanned is disposed;
b) a plurality of first optic fibers disposed in an arc around said opening below said tabletop, said optic fibers being directed toward said opening;
c) a second optic fiber having a first end coupled to a laser beam and a second end directed toward and across said opening to define a slice plane such that said laser beam impinges on the breast in said opening and light exiting from the breast is picked up by said first optic fibers;
d) a plurality of optical detectors each operably associated with each respective first optic fibers, said first optic fibers being arranged such that said detectors are disposed below said scan plane;
f) said laser beam, said first optic fibers, said second optic fiber and said detectors are adapted to be orbited at the same time around the breast about an orbital axis through said opening; and
g) each of said detectors are configured to simultaneously detect light exiting the breast being scanned within the respective field-of-view of each first optic fiber.

14. A scanner as in claim 13, wherein said detectors are oriented to detect light in a vertical direction.

15. A scanner as in claim 13, wherein said second optic fiber is S-shaped.

16. A scanner as in claim 13, and further comprising:
a) a first lens coupled to an exit end of said second optic fiber;
b) a second lens coupled to an entry end of said second optic fiber; and
c) a plurality of lens each coupled to a respective entry end of each first optic fibers.

17. A scanner as in claim 13, wherein:
a) said first optic fibers have entry and exit ends; and
b) said entry ends are arranged in a series of arcs around said opening, each arc being disposed below a topmost arc.

18. A scanner for a laser imaging apparatus, comprising:
a) a tabletop having an opening in which a breast to be scanned is disposed;
b) a laser beam originating below said tabletop;
c) means for folding and directing said laser beam across said opening to impinge on the breast to define a scan plane;
d) means disposed below said scan plane for detecting said laser beam after passing through the breast;
e) means for restricting the field-of-view of said detecting means; and
f) said laser beam, said detecting means and said restricting means are adapted to be orbited at the same time around the breast about an orbital axis through said opening.
